(19) United States
(12) Patent Application Publication
(10) Pub. No.: US 2006/0153809 A1
(43) Pub. Date: Jul. 13, 2006

(54) Recombinant trail vectors and uses thereof

(76) Inventors: Ruian Xu, Hong Kong (HK); Dexian Zheng, Beijing (CN); Juan Shi, Beijing (CN); Yanxin Liu, Beijing (CN)

Correspondence Address: COOPER & DUNHAM, LLP, 1185 AVENUE OF THE AMERICAS, NEW YORK, NY, 10036, US

(21) Appl. No.: 11/330,450

(22) Filed: Jan. 12, 2006

Related U.S. Application Data
(60) Provisional application No. 60/644,782, filed on Jan. 13, 2005.

(51) Int. Cl. A61K 48/00; C12N 15/861

(52) U.S. Cl. 424/93.2; 435/456

(57) ABSTRACT

This invention provides a composition comprising a recombinant adeno-associated virus 2/5 (rAAV2/5) which encodes consecutive amino acids 114-281 of the amino acid sequence of the extracellular domain of tumor necrosis factor-related apoptosis-inducing ligand (TRAIL). This invention also provides a pharmaceutical composition comprising such composition and a pharmaceutically acceptable carrier. This invention provides a method for treating lung cancer in a subject. The invention further provides a kit comprising a pharmaceutical composition comprising a composition comprising a recombinant adeno-associated virus 2/5 (rAAV2/5) which encodes consecutive amino acids 114-281 of the amino acid sequence of the extracellular domain of tumor necrosis factor-related apoptosis-inducing ligand (TRAIL) and a pharmaceutically acceptable carrier, and instructions for use.

This application claims priority of U.S. Provisional Application No. 60/644,782, filed Jan. 13, 2005, the contents of which are hereby incorporated by reference into this application.

Throughout this application, various publications may be referenced. Full citations for these publications may be found at the end of the specification immediately preceding the claims. The disclosures of these publications in their entireties are hereby incorporated by reference into this application in order to more fully describe the state of the art as known to those skilled therein as of the date of the invention described and claimed herein.

### BACKGROUND OF THE INVENTION

Lung cancer is the most lethal cancer in the world. The average worldwide incidence of lung cancer is 37.5 per 100,000 persons, though this number varies greatly by country (Cai, et al., 2003; Greenlee et al., 2001). Most patients die of progressive metastatic disease despite advances in surgical oncology and the development of therapies (Siegfried et al., 1998, Cai et al., 2003). Current treatments in clinical trials yielded only very limited results. A great need exists for an effective therapy for this lethal disease. Gene therapy of cancer has shown to be applicable in several clinical situations and these strategies are already proving their full potential in cancer treatment.

TNF-related apoptosis inducing ligand (TRAIL/Apo-2L) is a typical member of the TNF ligand family that induces apoptosis through activating the death receptors (Mongkolsapaya et al, 1999; Hymowitz et al., 2000). In recent years, considerable attention has been focused on the potential benefits of TRAIL in cancer therapy. Many reports have demonstrated that TRAIL can induce apoptosis in various tumor cells, but not in most normal cells, and is relatively nontoxic in vivo (Ashkenazi et al., 1999; Wlczak et al., 1999). Studies using recombinant TRAIL protein have demonstrated that the extracellular domain of TRAIL was capable of inducing apoptosis (Ashkenazi et al., 1999; Wlczak et al., 1999). Furthermore, studies on different cancer cells revealed that combination treatment with chemotherapeutic or ionizing radiation strongly enhanced the cytotoxic effect of TRAIL in both TRAIL-sensitive and TRAIL-resistant cells, and was also capable of sensitizing cancer cells resistant to chemotherapeutic or ionizing radiation treatment (Keane et al., 1999; Gibson et al., 2000). As with other drugs, the delivery of soluble TRAIL as a therapeutic agent for routine clinical use faces certain technical challenges. First, relatively large amounts of soluble TRAIL are required to inhibit tumor growth (Ashkenazi et al., 1999). Second, instability and distribution kinetics must be considered, because soluble TRAIL is cleared from the blood within 5 hours (Ashkenazi et al., 1999). Several attempts have been made to use TRAIL as the target protein of gene therapy because of its potential in cancer therapy. It has recently been demonstrated that transfer of the TRAIL gene or the green fluorescent protein fusion (GFP)/TRAIL gene could induce apoptosis and the apoptotic bystander effect in various human cancer cells in vitro (Griffith et al., 2000; Voelkel-Johnson et al., 2002), and in vivo in the case of human glioblastoma, breast carcinoma xenografts (Lee et al., 2002), human prostate tumor xenograft (Griffith et al., 2001), and human colon tumor xenograft (Kagawa et al., 2001). However, the virus vectors currently used in TRAIL gene therapy are adenovirus. These vectors may do harm to the hepatic cells through the innate and cell-mediated immune responses. By contrast, recombinant adeno-associated virus (AAV) is not associated with any human disease and the weak immunogenicity of AAV enables a safe vector administration into a target-tissue for several times as shown in several clinical studies (Kay et al., 2000). In addition, AAV has a high transduction capacity in primary human cell (Rohr et al., 2002). Here, the tropism of different “pseudotyped” version of rAAV2 (rAAV2/1, rAAV2/2, rAAV2/5, rAAV2/6, rAAV2/8) for a human lung adenocarcinoma cell line A549 is examined and rAAV2/5 is identified as a promising type for AAV mediated gene transfer to lung cancer. A recombinant AAV2/5 expressing TRAIL₁₁₄₋₂₈₁ was constructed to study TRAIL₁₁₄₋₂₈₁ expression in transfected A549 cells and anti-tumor activities in two experimental transplantic lung cancer models in vivo. Results detected secreted expression form of TRAIL₁₁₄₋₂₈₁ mediated by the recombinant rAAV2/5-TRAIL₁₁₄₋₂₈₁ shortly after cell infection. Moreover, a significant regression of tumor was observed after transduction with rAAV2/5-TRAIL in two human lung adenocarcinoma xenograft models, suggesting that the recombinant vector of rAAV2/5-TRAIL may be a potential approach for lung cancer treatment.

The major concern with the application of TRAIL in the treatment of tumors in vivo is its controversial role in hepatic cell death (Shi et al., 2003; Jo et al., 2000; Ichikawa et al., 2001). Interestingly, TRAIL studies showing hepatotoxicity are all either with the full-length membrane-bound form of the protein (Ichikawa et al., 2001) or, if soluble, exogenous sequence tags (Jo et al., 2000). A histidine tagged TRAIL has been shown to have an altered protein conformation, reduced stability, decreased solubility and hepatotoxicity (Lawrence et al., 2001). However, the same protein without the histidine tag was able to trimerize adequately, giving it biological activity and neoplastic cell toxicity, with little or no evidence of toxicity to primary human hepatocytes in vitro (Lawrence et al., 2001). These observations are consistent with the absence of toxicity in our TRAIL protein study. Even the addition of 1 μg/ml recombinant soluble TRAIL prepared in our laboratory did not induce apoptosis in freshly isolated human hepatocytes in culture. It would therefore appear that soluble TRAIL lacks the hepatotoxicity that is associated with other forms of TRAIL, but has the ability to induce apoptosis in a variety of tumor cell lines, including the A549 lung adenocarcinomas. The hepatotoxicity of TRAIL may be affected not only by the nature of TRAIL, but also by the physiological condition of the hepatocytes and other interacting factors, such as innate and adaptive immune responses to the vector. Therefore, the adenovirus-induced upregulation of TRAIL receptor DR5 may be an important contributory factor in the reported hepatotoxicity of adenovirus encoded TRAIL (Zhang et al., 2002).

The present invention provide the first report of rAAV mediated delivery and expression of soluble TRAIL₁₁₄₋₂₈₁, demonstrating that intratumoral or intratracheal injection of rAAV2/5-sTRAIL result in the presence of the trimeric form of sTRAIL in sera, a statistically significant reduction in the rate of tumor growth, and the prolonged survival of tumor bearing mice. The rAAV2/5-sTRAIL administration did not cause any detectable toxicity either in the primary human hepatocytes in culture, or to any of the examined organs of the transduced mice in vivo, suggesting that AAV2/5 mediated soluble TRAIL gene therapy may provide a feasible and effective form of treatment for lung cancer.

### SUMMARY OF THE INVENTION

This invention provides a composition comprising a recombinant adeno-associated virus 2/5 (rAAV2/5) which encodes consecutive amino acids 114-281 of the amino acid sequence of the extracellular domain of tumor necrosis factor-related apoptosis-inducing ligand (TRAIL).

This invention also provides a pharmaceutical composition comprising a composition comprising a recombinant adeno-associated virus 2/5 (rAAV2/5) which encodes consecutive amino acids 114-281 of the amino acid sequence of the extracellular domain of tumor necrosis factor-related apoptosis-inducing ligand (TRAIL) and a pharmaceutically acceptable carrier.

This invention provides a method for treating lung cancer in a subject comprising administering to the subject a therapeutically effective amount of a pharmaceutical composition comprising a composition comprising a recombinant adeno-associated virus 2/5 (rAAV2/5) which encodes consecutive amino acids 114-281 of the amino acid sequence of the extracellular domain of tumor necrosis factor-related apoptosis-inducing ligand (TRAIL) and a pharmaceutically acceptable carrier

The invention further provides a kit comprising a pharmaceutical composition comprising a composition comprising a recombinant adeno-associated virus 2/5 (rAAV2/5) which encodes consecutive amino acids 114-281 of the amino acid sequence of the extracellular domain of tumor necrosis factor-related apoptosis-inducing ligand (TRAIL) and a pharmaceutically acceptable carrier, and instructions for use.

### BRIEF DESCRIPTION OF THE FIGURES

FIG. 1A-D—rAAV mediated transduction of A549 cells and sTRAIL mediated induction of apoptosis. (A) Flow cytometric analysis of transduction efficiency of human lung adenocarcinoma cell line A549 with different AAV vectors (60 h after infection at an MOI of 5×10⁴ particles/cell, n=3). (B) A549 cells infected with the indicated vectors (MOI=5×10⁴) were examined by fluorescent microscopy after Hoechst staining (1,200× magnification). (C) Cell viability determined by MTT assay in A549 cells (●, rAAV2/5-sTRAIL; ▴, rAAV2/5-eGFP) and in HEK293 cells (▪, rAAV2/5-sTRAIL; ♦, rAAV2/5-eGFP; n=3). (D) Western-blot analysis of A549 cells infected with rAAV2/5-sTRAIL (MOI=5×10⁴ and harvested after 72 h (Lane 1: rAAV2/5-eGFP, lane 2: rAAV2/5-sTRAIL).

FIG. 2—Structure of rAAV2/5-sTRAIL vector

FIG. 3A-D—Transgene expression and cytotoxicity in primary human hepatocytes in culture. Human hepatocytes were infected at an MOI of 2×10⁵ with rAAV2/5-eGFP (A,C) or rAAV2/5-sTRAIL (B, D) and examined by fluorescent (A) or light microscopy 5 days later (100× magnification).

FIG. 4A-B—Intratumoral injection of rAAV2/5-sTRAIL suppresses the growth of s.c. A549 tumors in nude mice. A549 cells (5×10⁶) were injected in the s.c. tissues of the left dorsal flank of nude mice. (A) Tumor growth in the A549 tumor bearing mice that were injected with either rAAV2/5-sTRAIL (▴), rAAV2/5-eGFP (▪), or the carrier PBS (♦) (n=7, each). (B) Representative transverse sections of tumors excised 45 days after vector injection, stained with hematoxylin-eosin (100× magnification). Tumors injected with rAAV2/5-sTRAIL present clear evidence of cell death, but not those in PBS or rAAV2/5-eGFP-injected.

FIG. 5A-D—Intratumoral injection of rAAV2/5-sTRAIL induces tumor cell apoptosis and inhibits tumor vascularization. Histological examination of the tumors was carried out 45 days after intratumoral injection of the indicated vectors or carrier PBS. (A) TUNEL staining of the tumor sections demonstrate a much larger fraction of TUNEL positive cells (green) in the rAAV2/5-sTRAIL-injected than in the injected with the carrier PBS (200× magnification). (B) Immunochemical detection of TRAIL positive cells, using a mouse anti-TRAIL antibody (brown signals, 400× magnification). (C) Tissue sections stained with anti-CD31 (brown signals, 200× magnification). (D) Blood vessels stained with anti-CD31 (see panel C) were quantified by densitometry.

FIG. 6A-E—Intratracheal administration of rAAV2/5-sTRAIL suppresses orthotopic A549 tumors in nude mice. Tumor cells (1×10⁶) were implanted into the lung parenchyma through the intercostals space. One week later, animals were randomized into three groups and subjected to intratracheal administration of 5×10¹¹ particles of rAAV2/5-sTRAIL, rAAV2/5-eGFP or the carrier PBS (n=7). Mice were sacrificed on day 45. A substantial reduction in the number of tumor nodules was visible in the lungs of the rAAV2/5-sTRAIL-transduced animals, but not in those injected with rAAV2/5-eGFP or the carrier PBS (A and C). Histological examination of hematoxylin-eosin stained lung tissue sections (100× magnification) (B). Panel C data represents the means±SD of the number of tumor nodules in the lungs (n=5). (D) Immunochemical detection of sTRAIL protein in the lung tissue was examined 38 days after rAAV2/5-sTRAIL transduction (brown signals, 800× magnification). (E) Western blot analysis of serum proteins 38 days after the intratracheal transduction with rAAV2/5-sTRAIL. The trimeric form of sTRAIL presents in the serum of the rAAV2/5-sTRAIL group (Lanes 2-4), but not in the rAAV2/5-eGFP group (Lane 1).

FIG. 7A-C—sTRAIL mediated inhibition of angiogenesis and the increased survival of tumour bearing mice. (A) Tissue sections stained with anti-CD34 for the detection of tumor vasculature (brown signals, 400× magnification). The PBS group sacrificed on day 45 (1), and the rAAV2/5-sTRAIL group sacrificed either on day 45 (2) or day 60 (3). (B) The intratracheal introduction of rAAV2/5-sTRAIL prolongs the survival of mice with orthotopic A549 lung tumors. The mice received 5×10¹¹ particles of rAAV2/5-sTRAIL, the empty rAAV2/5 vector, or PBS, 7 days after the orthotopic implantation of 1××10⁶ A549 cells. The animals were monitored for euthanasia (see Materials and Methods) (n=10, P<0.01). (C) Hepatotoxicity of rAAV2/5-sTRAIL in vivo. Serum samples were collected 30 days after the intratracheal introduction of 1×10¹² particles of rAAV2/5-sTRAIL (1) or PBS (2) to non-tumor bearing mice.

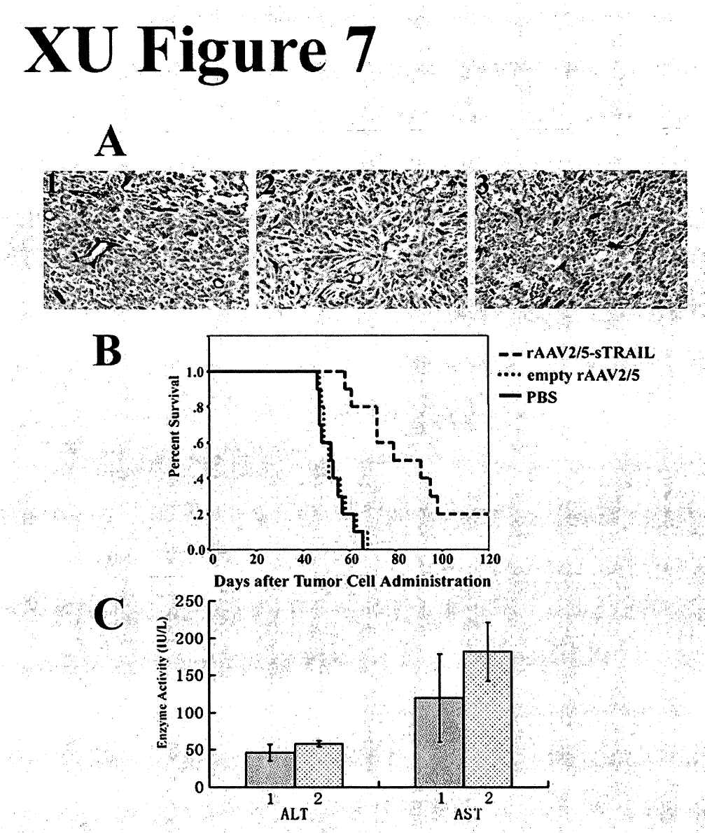

### DETAILED DESCRIPTION OF THE INVENTION

This invention provides a composition comprising a recombinant adeno-associated virus 2/5 (rAAV2/5) which encodes consecutive amino acids 114-281 of the amino acid sequence of the extracellular domain of tumor necrosis factor-related apoptosis-inducing ligand (TRAIL).

The consecutive amino acids 114-281 of the amino acid sequence of the extracellular domain of TRAIL may be a rat TRAIL sequence or a human TRAIL sequence.

This invention also provides a pharmaceutical composition comprising a composition comprising a recombinant adeno-associated virus 2/5(rAAV2/5) which encodes consecutive amino acids 114-281 of the amino acid sequence of the extracellular domain of tumor necrosis factor-related apoptosis-inducing ligand (TRAIL) and a pharmaceutically acceptable carrier.

This invention provides a method for treating lung cancer in a subject comprising administering to the subject a therapeutically effective amount of a pharmaceutical composition comprising a composition comprising a recombinant adeno-associated virus 2/5 (rAAV2/5) which encodes consecutive amino acids 114-281 of the amino acid sequence of the extracellular domain of tumor necrosis factor-related apoptosis-inducing ligand (TRAIL) and a pharmaceutically acceptable carrier. In one embodiment, the method further comprises administering chemotherapeutic agents or irradiation, thereby increasing the apoptotic effect of TRAIL.

In one embodiment of the invention, the composition transduces lung tumor cells. The transduction results in the suppression or inhibition of tumor growth. In another embodiment of the invention, the transduction results in secretion of soluble TRAIL (sTRAIL) and induces apoptosis of the lung tumor cells. In yet another embodiment of the invention, the transduction results in high expression of soluble TRAIL (sTRAIL) in vivo.

In one embodiment of the invention, the pharmaceutical composition is administered invasively or non-invasively. In another embodiment, the subject is human.

The invention further provides a kit comprising a pharmaceutical composition comprising a composition comprising a recombinant adeno-associated virus 2/5 (rAAV2/5) which encodes consecutive amino acids 114-281 of the amino acid sequence of the extracellular domain of tumor necrosis factor-related apoptosis-inducing ligand (TRAIL) and a pharmaceutically acceptable carrier, and instructions for use.

Experimental Details

A. Synopsis

Lung cancer is currently the leading cause of cancer death in the world. Here the potential of recombinant adeno-associated virus type-5 (rAAV-5) vectors mediated TRAIL for lung cancer gene therapy is elucidated. The rAAV-5 strain is among the most efficient transducers of human lung adenocarcinoma A549 cell line among rAAV strains. An AAV2/5 vector encoding extracellular domain (encoding 114˜281 peptide) of TRAIL from a normal Chinese peripheral lymphocyte was constructed. Cell growth of A549 cells were significantly reduced in a time-dependent manner in comparison with control vectors after transduction with rAAV2/5-TRAIL. The reduction of tumor cell growth was associated with increased apoptosis. In two Xenograft mouse models of human lung cancer, rAAV2/5-TRAIL gene therapy resulted in significant growth inhibition. TRAIL₁₁₄₋₂₈₁ was also highly expressed in vivo and secreted into the serum in trimeric active form. Exogenous TRAIL induced apoptosis of cancer cells but not apoptosis of normal cells. Successful inhibition of lung cancer growth following transduction with rAAV2/5-TRAIL₁₁₄₋₂₈₁ vector underlines the potential role in cancer gene therapy.

B. Materials and Methods

Animals: Surgical procedures and care of animals, approved by the Ethics Committee of the University of Hong Kong, were performed according to institutional guidelines. Male, 5-6 weeks old BALBc nude mice were housed at a constant temperature and supplied with laboratory chow and water ad libitum on a 12 h dark/light cycle.

Construction of the rAAV-sTRAIL and rAAV-eGFP Vectors: The sTRAIL cDNA insert encoding TRAIL amino acids 114-281 was amplified from TRAIL cDNA as previously reported (17). The cytomegalovirus (CMV) enhancer/chicken β-actin promoter, the inserted genes and polyA sequences were inserted between the ITRs using appropriate restriction enzymes. To boost transgene expression, a woodchuck hepatitis B virus post-transcriptional regulatory element was inserted into both constructs immediately after the expression cassette (15). DNA sequence analysis confirmed the integrity of the cDNA constructs and the vector.

Pseudotyping: Different pseudotypes of rAAV were generated by standard production and cesium chloride density sedimentation purification protocols, using a slightly modified three plasmid helper-virus free packaging method (18). The rAAV viral genome titer was quantified by real-time PCR analysis (AB Applied Biosystem, Foster City, Calif.).

Analysis of Transgene Expression in vitro: HEK 293 or A549 cells were cultured in the complete medium before the addition of rAAV at a dose of 10⁴ particles/cell in culture medium supplemented with 2% FBS for 5 h, followed by incubation in complete medium containing 10% FBS. The efficiency of infection with rAAV-eGFP was analysed by fluorescence microscopy. The culture media of the transduced cells were collected and concentrated by vacuum desiccation for analysis of the secreted TRAIL.

Analysis of Apoptosis and Cell Death: Cells, fixed with 4% paraformaldehyde (PFA) in PBS for 20 min, were stained with Hoechst 33258 at 1 μg/ml in PBS for 15 minutes. Apoptosis was visualized with fluorescence microscopy and cytotoxicity was quantified by 3-(4,5-dimethylthiazole-2-yl)-2,5-biphenyl tetrazolium (MTT) assay (Sigma). Microtiter 96-well plates were seeded with 1×10⁴ A549 or HEK293 cells/well and cultured for at least 12 h at 37° C., followed by infection with rAAV2/5-sTRAIL or rAAV2/5-eGFP vectors at 10⁴ particles/cell. After 24, 36, 48, 72 h or longer, 10 μl of 5 mg/ml MTT was added to each well and incubated for 4 h at 37° C. The medium was removed and the MTT crystals were solubilized in 100 μl 0.04N HCl/isopropanol. A SPECTRA MAX 340 microplate reader (Molecular Devices, Menlo) was used to quantify the optical density in each sample.

Subcutaneous Tumor Xenografts and Assessment of Growth: The A549 cells (5×10⁶) were injected in the s.c. tissue of the left dorsal flank of 7-week-old male nude mice. Tumor growth was monitored three times a week and volume (V) was calculated using the formula V=½×length×[width]². When tumor size reached approximately 50 mm³ (about 10 days post inoculation), the animals were randomized into three groups and vectors were administered by intratumoral injection. Groups 1 and 2 received rAAV2/5-sTRIAL or rAAV2/5-eGFP, respectively and group 3 mice received PBS as control. Experiments were terminated once the tumors reached 500-600 mm³ in the control PBS injected groups (around 55 days after tumor cells inoculation).

Orthotopic Lung Cancer Model and Assessment of Growth: Orthotopic implantation of A549 cells was performed as described by Yamaura et al (19). In short, A549 cells (5×10⁷/ml) were suspended in PBS containing 1 mg/ml of Matrigel (BD Biosciences, Bedford, Mass.). The left chests of anesthetized mice were incised and 20 μl aliquots of the cell suspension were injected into the lung parenchyma through the intercostals space (approximately 3 mm depth) and the incisions were closed. Only single-cell suspensions with >90% viability were injected. One week later animals were randomized into three groups and the vectors were delivered by intratracheal administration of 30 μl PBS or 5×10¹1 particles of either rAAV2/5-sTRAIL or rAAV2/5-eGFP in PBS. Mice were sacrificed 45 days after tumor implantation. Lungs were excised and tumour formation and the metastatic features of A549 were investigated in 3 randomly selected hematoxylin-eosin stained sections. For survival studies, other groups of mice were treated as above, weighed three times weekly and assessed for indicators of tumor growth and of a moribund state. Animals were euthanized if they developed two or more of the following: gross ascites, palpable tumor burden>2 cm, dehydration, emaciation, or weight loss>20% of initial body weight. Survival was monitored for 4 months.

Analysis of Gene Expression and Microvessel Density (MVD): The primary antibodies, TRAIL, caspase-3 and caspase-8 were from Santa Cruz Biotechnology (Santa Cruz, Calif.). CD31 and CD34 from Pharmingen (San Diego, Calif.). Western blot analysis, histology, immunohistochemical staining, ELISA and TUNEL assays were carried out as previously described (20). MVD was assessed by the hot spot method (21).

Statistical Analysis: Log rank tests were performed for survival data. For other data, results were expressed as mean values±standard deviation (s.d.). Student's t test was used to evaluate statistical significance. P values were considered to be statistically significant when <0.05.

C. Results

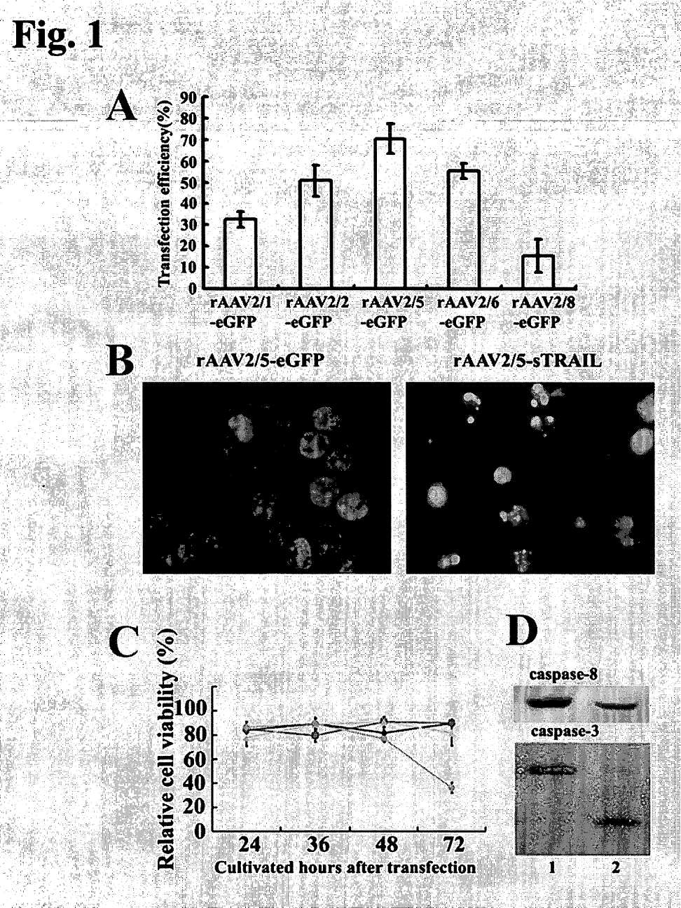

To investigate the transduction efficiency of different rAAV serotypes, high-titer rAAV2/1, rAAV2/2, rAAV2/5, rAAV2/6 and rAAV2/8 vectors expressing eGFP were used to infected the A549 human lung cancer cell line at a multiplicity of infection (MOI) of 5×10⁴. Flow cytometric analysis demonstrated transduction rates to be 32.62±3.69% for rAAV2/1, 50.78±7.4% for rAAV2/2, 70.44±6.74% for rAAV2/5, 55.48±3.48% for rAAV2/6 and 18.56±7.80% for rAAV2/8 (see FIG. 1A). Therefore, rAAV2/5 was identified as the most efficient of the examined pseudotypes for the in vitro transduction of A549 cells.

Induction of A549 Tumor Cell Apoptosis by rAAV2/5-sTRAIL To determine the apoptosis-inducing activity of rAAV2/5-sTRAIL, A549 cells were infected with rAAV2/5-sTRAIL or rAAV2/5-eGFP, stained with Hoechst 33258, and microscopically examined for evidence of apoptosis (FIG. 1B). Condensed nuclei, a characteristic of apoptosis, were present in substantially greater numbers in the rAAV2/5-sTRAIL, but not the rAAV2/5-eGFP, infected cells. Similarly, analysis of toxicity by the MTT assay demonstrated the induction of toxicity in the rAAV2/5-sTRAIL, but not the rAAV2/5-eGFP infected cells. There was also no evidence of toxicity in the transduced HEK293 cells (FIG. 1C). To further test apoptosis, A549 cells infected with rAAV2/5-sTRAIL (MOI=5×10⁴) were subjected to Western blot analysis, revealing the presence of activated caspase 3 and decreased levels of caspase-8 precursor protein (FIG. 1D).

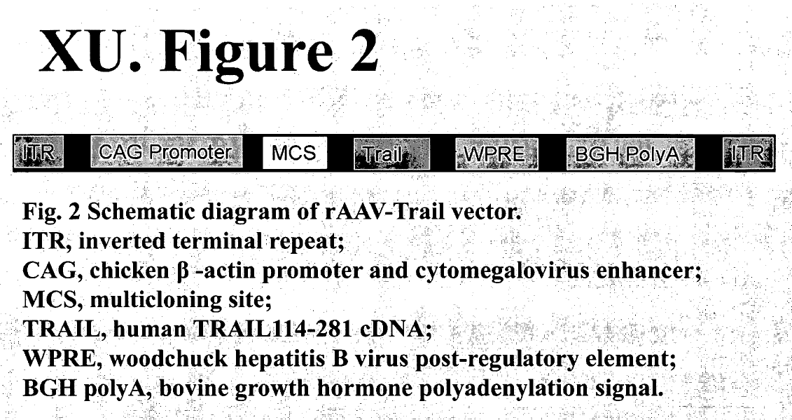

Next we tested whether the encoded TRAIL could be secreted into the medium. ELISA showed a peak value of 282.93+69.71 ng/ml at 48 hours in the culture media of rAAV2/5-sTRAIL transduced HEK293 cells. In contrast the maximal sTRAIL concentration was only 97.63+41.86 ng/ml in the similarly transduced A549 cells, and this level was reached 24 hours post-infection. The lower maximal expression level and the absence of further increases are likely to be due to the toxic effects of TRAIL on A549 cells. To test the potential toxicity of rAAV2/5-sTRAIL on normal cells, primary human hepatocytes were infected with rAAV2/5-eGFP or rAAV2/5-sTRAIL. There was no apparent evidence of rAAV2/5-sTRAIL induced toxicity in these cells, even when the vector dose was increased 4 fold to an MOI of 2×10⁵ and the duration extended to 5 days (FIG. 2). Together, these results show that sTRAIL is secreted from rAAV2/5-sTRAIL infected cells and that it can induce apoptosis in the human lung cancer A549 cells, but not in the normal human hepatocytes.

Suppression of Subcutaneous Tumor Growth

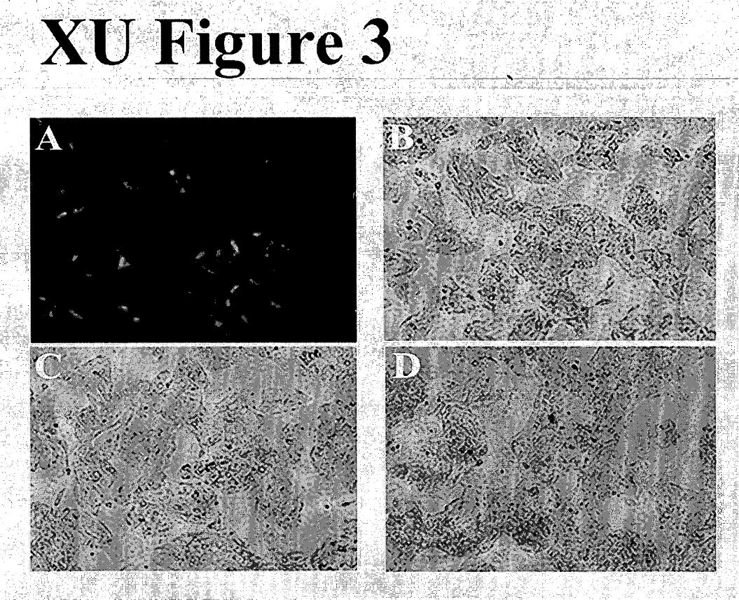

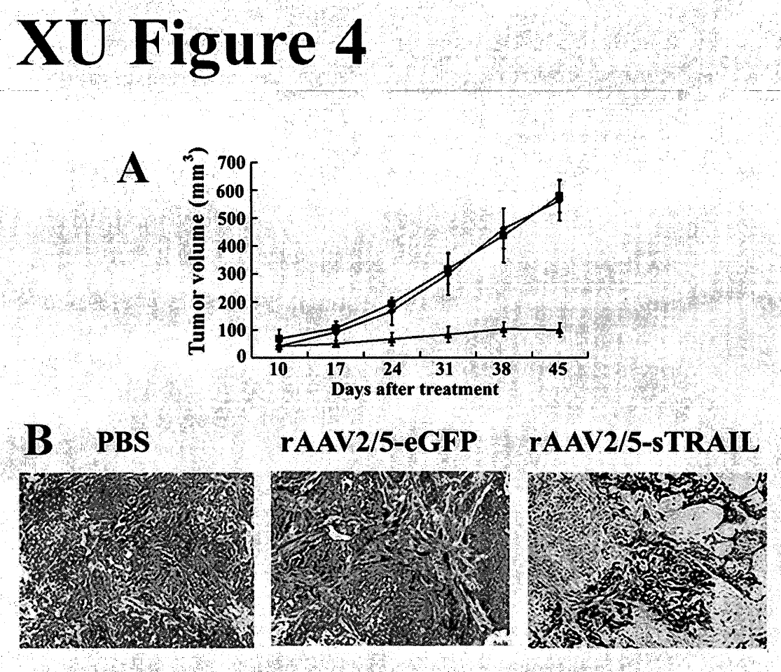

To analyze the therapeutic potential of rAAV2/5-sTRAIL, nude mice were subcutaneously injected with 5×10⁶ A549 cells in the left dorsal flank. 10 days later (approx. tumour volume 50 mm³), animals were randomized into three groups and received an intratumoral injection of 3×10¹¹ particles of rAAV2/5-sTRAIL, rAAV2/5-eGFP or the carrier PBS. The monitoring of tumour growth for approximately 7 weeks revealed substantial growth reduction in the rAAVs/5-sTRAIL injected tumors (FIG. 3A), but not in the rAAV2/5-eGFP or PBS control groups. Histological examinations confirmed smaller tumors in the rAAV2/5-sTRAIL group, with large areas of cell death and substantially reduced numbers of scattered tumor cells (FIG. 3B). To determine whether transduction with rAAV2/5-sTRAIL led to tumor cell apoptosis, TUNEL assays were carried out. In the initial few days, there were no significant differences either in tumour volume (FIG. 3A) or level of apoptosis between the TRAIL and the control groups (data not shown). However, significantly higher levels of apoptotic cell death was detectable in the sTRAIL transduced tumours on day 45 (FIG. 4A). Immunohistochemical studies also provided clear evidence of TRAIL expression 45 days after intratumoral injection of rAAV2/5-sTRAIL, but not in the PBS injected tumors (FIG. 4B). Analysis of CD31 expression in the tumor sections demonstrated significant reductions in the MVD in the rAAV2/5-sTRAIL treated animals as compared to the two control groups (P<0.05) (FIGS. 4C and 4D).

Suppression of Orthotopic Lung Tumor Growth Following Intratracheal Administration of rAAV viruses

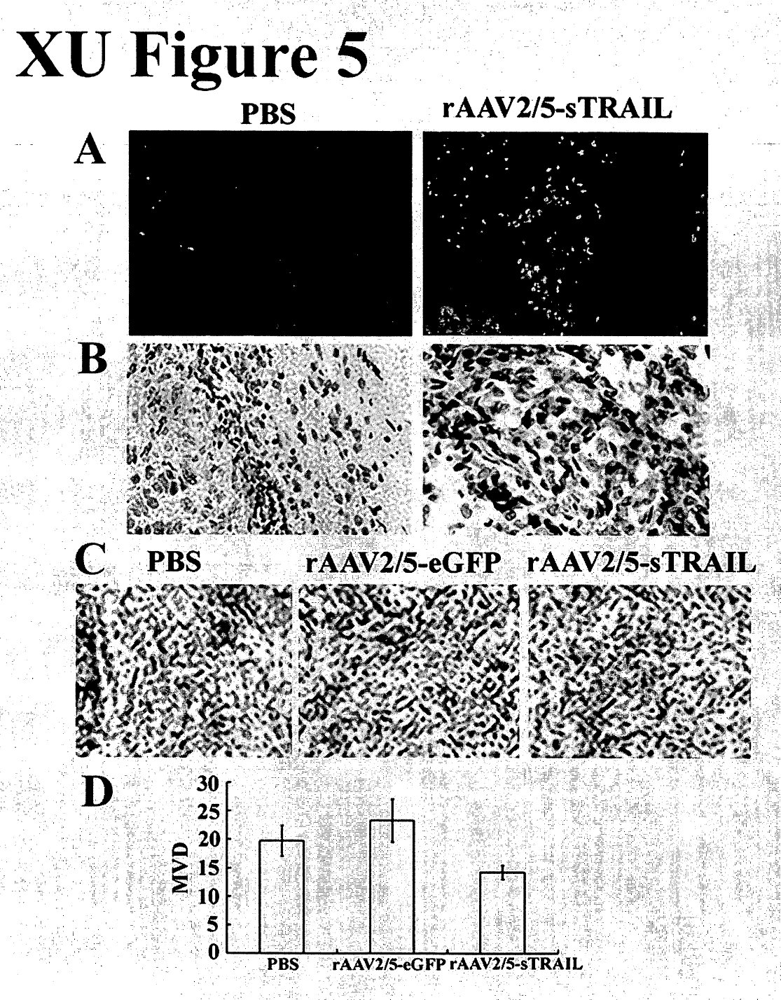

To better evaluate the anti-tumor activity of rAAV2/5-sTRAIL in lung cancer, 1×10⁶ A549 cells were injected into the lung parenchyma through the intercostals space. One week later, the mice were randomized into three groups and 5×10¹¹ particles of rAAV2/5-sTRAIL, rAAV2/5-eGFP or the carrier PBS were delivered by intratracheal administration. Mice were sacrificed 45 days after tumor implantation. Gross macroscopic examination of the chest cavity demonstrated the presence of tumor nodules on both the left and right lobes of the lung, particularly in the control groups (FIG. 5A). There was also clear evidence of heart metastases in 30% of the PBS and 20% of the rAAV2/5-eGFP treated mice (data not shown). Microscopic examination of hematoxylin and eosin stained lung tissue sections confirmed extensive metastatic disease in the control groups, but substantially reduced tumor presence in the lung tissue of the rAAV2/5-sTRAIL transduced animals (FIG. 5B). This was consistent with the macroscopically visible number of lung tumour nodules on day 45: 61+12 and 58+8 in the PBS and rAAV2/5-eGFP groups, compared to 20+12 in the rAAV2/5-sTRAIL treated mice (P<0.01 for either the PBS or rAAV2/5-eGFP groups, FIG. 5C). By day 60 the number of lung tumor nodules had increased further in all groups, including the TRAIL treated mice (increased from 20+12 on day 45 to 33+15 on day 60). However, this was still significantly lower than the number of nodules in the PBS (P=0.017) or the rAAV2/5-eGFP treated groups (P=0.015).

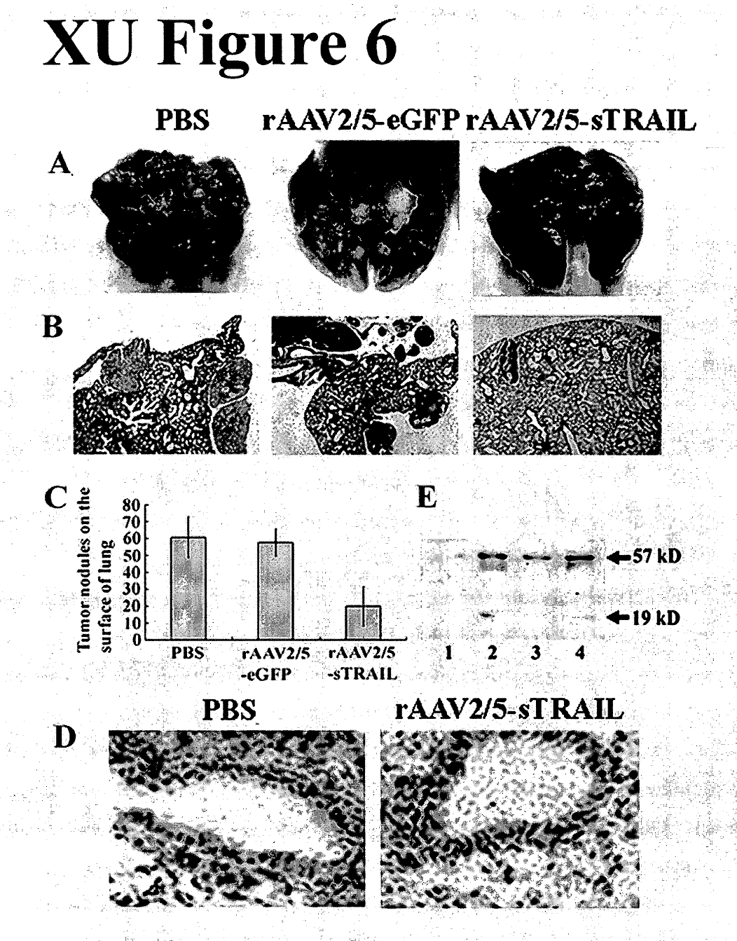

The expression of TRAIL in lung tissue sections isolated from the rAAV2/5-sTRAIL transduced tumors was confirmed by immunohistochemistry. The highest levels of TRAIL expression were in the airway epithelial cells (FIG. 5D). There was also clear evidence of the presence of biologically active trimeric form of TRAIL in the sera of the rAAV2/5-sTRAIL transduced animals (FIG. 5E), corresponding to a circulating serum level of 114+29 ng/ml at 38 days after the intratracheal administration of 5×10¹¹ particles of rAAV2/5-sTRAIL. Analysis of CD34 expression also revealed a statistically significant reduction in the microvessel density in the rAAV2/5-sTRAIL treated mice on day 45 after tumor cell administration (P<0.05, FIG. 6A) but this reduction decreased and was no longer significant by day 60 (data not shown).

rAAV2/5-sTRAIL Improves the Survival Rate of Mice with Lung Metastases

The orthotopic lung cancer model was also used to investigate the effect of rAAV2/5-sTRIAL infection on survival. Initial mortality began on day 46 for the PBS and day 47 for the rAAV2/5-eGFP treated group, whereas the first mortality in the rAAV2/5-TRAIL treated mice was on day 60. The median survivals were 52 and 51 days for the PBS and rAAV2/5-eGFP groups, compared to a median survival of 79 days for the TRAIL treated mice. All mice in the control groups had to be sacrificed by 68 days, where as 20% of the TRAIL treated mice survived for at least 120 days (FIG. 6B).

Examination of Toxicity of rAAV2/5-TRAIL In Vivo

The toxicity of rAAV2/5-sTRAIL treatment was examined in non-tumor bearing mice following the intratracheal injection of 1×10¹² rAAV2/5-sTRAIL. The serum levels of alanine transaminase (ALT) and aspartate transaminase (AST) were measured 30 days after transduction. Both AST and ALT levels were within the normal range (FIG. 6C) and histopathological studies showed no obvious lesions in the lung, liver, spleen or kidney. In addition, body weight, gross appearance and behavior provided no signs of systemic toxicity.

C, Discussion

Resistance to chemotherapy or radiation, propensity to metastasize, the difficulty in early diagnosis and the risk of cell dissemination during primary tumor surgery (22), are all factors contributing to poor prognosis in solid tumours. One advantage of gene therapy over protein administration is the continuous production of relatively large amounts of the protein for extended periods of time. Some of the most efficient vehicles for the transfer and expression of therapeutic genes, including TRAIL, are adenovirus based vectors (11-13). However, immunological responses to the adenovirus vectors result in the immune mediated clearance of the transduced cells, hence reduced duration and level of expression of such therapeutic gene products. Furthermore, the presence of these immunological reactions against the virus limit the repeat administration of adenovirus based vectors (23, 24). The reduced immunogenicity of AAV-based vectors provides distinct advantages in respect of each of these limitations, prolonging expression and reducing the required frequency of therapeutic interventions. Nevertheless, despite some success using AAV2-based vectors in the lung (25), the luminal introduction of such vectors have been largely disappointing (26). This may be in part due to poor tropism of AAV2 for airway epithelial cells. Alternative AAV pseudotypes can be generated by the packaging of AAV2 genome into capsids from other serotypes, allowing easy alteration of the vector pseudotype. Vectors based on AAV5 or 6 have been shown to enter from the apical side and more efficiently transduce airway epithelial cells (25). Capsid proteins from AAV5 have also been shown to bind to sialic acid (27), a component of the putative specific receptor present on the apical surface of airway epithelial cells. Consistent with these observations, the rAAV2/5 generated in the present studies proved to be efficient vehicles for the delivery and expression of TRAIL in both mouse airway epithelia and in the A549 human lung adenocarcinoma cells. As a consequence, high levels of TRAIL protein was readily detectable both at the site of vector administration and in the serum of these animals for at least 38 days after a single intratracheal administration of rAAV2/5-sTRAIL. Detected serum levels of TRAIL (114+29 ng/ml) were similar to the levels that could be detected 2 hours after a single i.v. injection of 10 mg/kg recombinant TRAIL protein to nude mice (9). A tet- or rapamycin-dependent regulatory system has been developed for AAV mediated delivery and inducible expression of transgene (28). The regulated expression of TRAIL is likely to be an important requirement for the clinical application of TRAIL based gene therapy. Such a regulated TRAIL expression system would reduce the risk of toxicity due to long-term expression.

The major concern with the application of TRAIL in the treatment of tumors in vivo is its controversial role in hepatic cell death (5, 6, 9, 29-31). Interestingly, the recombinant TRAIL studies that show hepatotoxicity are all either with the full-length membrane-bound form of the protein (31) or, if soluble, exogenous sequence tags (30). A histidine tagged has been shown to have an altered protein conformation, reduced stability, decreased solubility and hepatotoxicity (32). However, the same protein (soluble TRAIL1 14 281) without the histidine tag was able to trimerize adequately, giving it biological activity and neoplastic cell toxicity, with little or no evidence of toxicity to primary human hepatocytes in vitro (32). These observations are consistent with the absence of toxicity in the present study. Even the addition of 1 μg/ml recombinant soluble TRAIL prepared in our laboratory did not induce apoptosis in freshly isolated human hepatocyte in culture (data not shown). It would therefore appear that lacks the hepatotoxicity that is associated with other forms of TRAIL, but the ability to induce apoptosis in a variety of tumor cell lines, including the A549 lung adenocarcinomas used in the present study. The hepatotoxicity of TRAIL may be affected not only by the nature of TRAIL, but also by the physiological condition of the hepatocytes and other interacting factors, such as innate and adaptive immune responses to the vector. Therefore, the adenovirus induced upregulation of TRAIL receptor DR5 may be an important contributory factor in the reported hepatotoxicity of adenovirus encoded TRAIL (33).

Although TRAIL has been shown to induce apoptosis in various cancer cells, there is discrepancy about the sensitivity of HEK293 cells to TRAIL. Some studies have found HEK293 cells to be sensitive to TRAIL toxicity (34), while others suggest the resistance of these cells to TRAIL induced apoptosis (35). In the present study we could detect no apoptosis in the rAAV2/5-sTRAIL transduced HEK293 cells for about 4 days. The effect of soluble TRAIL is likely to be dose- and/or conformation-dependent for different cell lines. Cell-line differences in receptor status and effector pathways may also account for different responses to TRAIL. The present study shows that the in vivo transduction of A549 tumors with rAAV2/5-sTRAIL resulted in decreased presence of endothelial cell (EC) markers CD31 and CD34, as revealed by histological examination of the transduced tumor sections. Regulation of the angiogenic process involves a delicate balance of factors to promote and inhibit neovascularization. Tumors appear to be able to disrupt this balance, resulting in neoangiogenesis and development of a microenvironment favoring tumor growth and metastasis (36). ECs express TRAIL receptors And Li et al. have demonstrated that isolated ECs from human umbilical veins or human dermal microvessels, expressing DR4 and DR5, are sensitive to TRAIL induced apoptosis (37). However, several other studies have shown the resistance of primary human Ecs to TRAIL induced apoprosis, and even greater survival and proliferation of these cells by TRAIL induced activation of the Akt and ERK pathways (38). Furthermore, adenovirus-mediated overexpression of MDA-7 appears to result in significant inhibition of growth in subcutaneous lung cancer tumors with decreased expression of CD31 and upregulation of TRAIL (39). We observed reduced vasculature in s.c. tumors and the initial but not in the later development of orthotopic tumors, even though tumour growth was still significantly inhibited. The responsible for the observed effects of TRAIL on survival and the proliferation of vascular ECs remain to be elucidated.

In summary, the present studies provide the first report of rAAV mediated delivery and expression of soluble TRAIL 114-28 1, demonstrating that intratumoral or intratracheal injection of rAAV2/5-sTRAIL result in the presence of the trimeric form of sTRAIL in sera, a statistically significant reduction in the rate of tumor growth, and the prolonged survival of tumor bearing mice. The rAAV2/5-sTRAIL administration did not cause any detectable toxicity either in the primary human hepatocytes in culture, or to any of the examined organs of the transduced mice in vivo, suggesting that AAV2/5 mediated soluble TRAIL gene therapy may provide a feasible and effective form of treatment for lung cancer.

### REFERENCES

1. Greenlee R T, Hill-Harmon M B, Murray T. Thun M. Cancer statistics CA cancer J Clin 2001; 51(1):15-36.
2. Giaccone G. Clinical impact of novel treatment strategies Oncogene 2002;21(45):6970-81.
3. Johnson B E, Grayson J, Makuch R W, et al. Ten-year survival of patients with small-cell lung cancer treated with combination chemotherapy with or without irradiation. J Clin Oncol 1990; 8 (3):396-401.
4. Wiley S R, Schooley K, Smolak P J, et al. Identification and characterization of a new member of the TNF family that induces apoptosis. Immunity 1995; 3 (6):673-82.
5. Ashkenazi A, Pai R C, Fong S, et al. Safety and anti-tumor activity of recombinant soluble Apo2 ligand. J Clin Invest 1999; 104 (2):155-162.
6. Walczak H, Miller R E, Ariail K, et al. Tumoricidal activity of tumor necrosis factor-related apoptosis-inducing ligand in vivo. Nat Med 1999; 5 (2): 157-63.
7. Kagawa S, He C, Gu J, et al. Antitumor activity and bystander effects of the tumor necrosis factor-related apoptosis-inducing ligand (TRAIL) gene. Cancer Res 2001; 61(8):3330-8.
8. Chinnaiyan A M, Prasad U, Shankar S, et al. Combined effect of tumor necrosis factor-related apoptosis-inducing ligand and ionizing radiation in breast cancer therapy. Proc Natl Acad Sci USA 2000; 97 (4):1754-9.
9. Kelley S K, Harris L A, Xie D, et al. Preclinical studies to predict the disposition of Apo2L/tumor necrosis factor-related apoptosis-inducing ligand in humans: characterization of in vivo efficacy, pharmacokinetics, and safety. J Pharmacol Exp Ther 2001;299(1):31-8.
10. Griffith T S, Anderson R D, Davidson B L, Williams R D, Ratliff T L. Adenoviral-mediated transfer of the TNF-related apoptosis-inducing ligand/Apo-2 ligand gene induces tumor cell apoptosis J Immunol 2000; 165 (5):2886-94.
11. Lee J, Hampl M, Albert P, Fine H A. Antitumor activity and prolonged expression from a TRAIL-expressing adenoviral vector Neoplasia 2002; 4 (4):312-23.
12. Griffith T S, Broghammer E L. Suppression of tumor growth following intralesional therapy with TRAIL recombinant adenovirus Mol Ther 2001; 4(3):257-66.
13. Sova P, Ren X W, Ni S et al. A tumor-targeted and conditionally replicating oncolytic adenovirus vector expressing TRAIL for treatment of liver metastases Mol Ther 2004; 9 (4):496-509.
14. Kay M A, Manno C S, Ragni M V, et al. Evidence for gene transfer and expression of factor IX in haemophilia B patients treated with an AAV vector. Nat Genet 2000; 24(3):257-61.
15. Xu R A, Janson C G, Mastakov M, et al. Quantitative comparison of expression with adeno-associated virus (AAV-2) brain-specific gene cassettes. Gene Ther 2001; 8(17):1323-32.
16. Gregorevic P, Blankinship M J, Allen J M et al. Systemic delivery of genes to striated muscles using adeno-associated viral vectors. Nat Med 2004 10(8):828-34.
17. Liu Y X, Zhu X, Ma Z Y Expression, purification of and biological activity of rsTRAIL in E. Coli. Chin Sci Bulletin 1999;44:1306-9.
18. Xiao W, Chirmule N, Schnell M A, Tazelaar J, Hughes J V, Wilson J M. Route of administration determines induction of T-cell-independent humoral responses to adeno-associated virus vectors. Mol Ther 2000; 1(4):323-9.
19. Yamaura T, Murakami K, Doki Y, et al. Solitary lung tumors and their spontaneous metastasis in athymic nude mice orthotopically implanted with human non-small cell lung cancer. Neoplasia 2000;2 (4):315-24.
20. Xu R A, Sun X, Tse Lai-yin, Chan P C, Xu S, Xiao W, Kung H F, Krissansen G W, Fan S T. Long term expression of angiostatin suppresses metastatic liver cancer in mice. Hepatology 2003, 37, 1451-1460.
21. Weidner N, Semple J P, Welch W R, Folkman J. Tumor angiogenesis and metastasis correlation in invasive breast carcinoma. N Engl J Med 1991;324(1):1-8.
22. Mori M, Mimori K, Ueo H, et al. Molecular detection of circulating solid carcinoma cells in the peripheral blood: the concept of early systemic disease. Int J Cancer 1996; 68(6):739-43.
23. Wilson J M. Adenoviruses as gene-delivery vehicles N Engl J Med 1996; 334 (18): 1185-7.
24. Kim T W, Chung H, Kwon I C, Sung H C, Jeong S Y. In vivo gene transfer to the mouse nasal cavity mucosa using a stable cationic lipid emulsion Mol Cells 2000; 10 (2): 142-7.
25. Moss R B, Rodman D, Spencer L T et al. Repeated adeno-associated virus serotype 2 aerosol-mediated cystic fibrosis transmembrane regulator gene transfer to the lungs of patients with cystic fibrosis: a multicenter, double-blind, placebo-controlled trial. Chest 2004; 125 (2):509-21.
26. Duan D, Yue Y Yan Z, Yang J, Engelhardt J F. Endosomal processing limits gene transfer to polarized airway epithelia by adeno-associated virus. J Clin Invest 2000; 105 (11):1573-87.
27. Walters R W, Yi S M, Keshavjee S, et al Binding of adeno-associated virus type 5 to 2,3-linked sialic acid is required for gene transfer. J Biol Chem 2001; 276 (23):20610-6.
28. Ye X, Rivera V M, Zoltick P. et al. Regulated delivery of therapeutic proteins after in vivo somatic cell gene transfer. Science 1999; 283(5398):88-91.
29. Shi J, Zheng D, Man K, Fan S T, Xu R. TRAIL: a potential agent for cancer therapy. Curr Mol Med 2003;3 (8):727-36.
30. Jo M, Kim T H, Seol D W, et al. Apoptosis induced in normal human hepatocytes by tumor necrosis factor-related apoptosis-inducing ligand. Nat Med 2000; 6 (5):564-7.
31. Ichikawa K, Liu W, Zhao L, et al. Tumoricidal activity of a novel anti-human DR5 monoclonal antibody without hepatocyte cytotoxicity. Nat Med 2001; 7 (8):954-60.
32. Lawrence D, Shahrokh Z, Marsters S, et al. Differential hepatocyte toxicity of recombinant Apo2L/TRAIL versions. Nat Med 2001; 7 (4):383-5.
33. Zhang H G, Xie J, Xu L, et al. Hepatic DR5 induces apoptosis and limits adenovirus gene therapy product expression in the liver. J Virol 2002; 76 (11):5692-700.
34. Gibson E M, Henson E S, Haney N, Villanueva J, Gibson S B. Epidermal growth factor protects epithelial-derived cells from tumor necrosis factor-related apoptosis-inducing ligand-induced apoptosis by inhibiting cytochrome c release. Cancer Res 2002; 62 (2):488-96.
35. Ozoren N, Kim K, Burns T F, Dicker D T, Moscioni A D, El-Deiry W S. The caspase 9 inhibitor Z-LEHD-FMK protects human liver cells while permitting death of cancer cells exposed to tumor necrosis factor-related apoptosis-inducing ligand. Cancer Res 2000; 60 (22):6259-65.
36. Addison C L, Arenberg D A, Morris S B, et al. The CXC chemokine, monokine induced by interferon-gamma, inhibits non-small cell lung carcinoma tumor growth and metastasis. Hum Gene Ther 2000; 11 (2):247-61.
37. Li, J H, Kirkiles-Smith N C, McNiff J M, Pober J S. TRAIL induces apoptosis and inflammatory gene expression in human endothelial cells. J Immunol 2003; 171 (3):1526-33.
38. Secchiero P, Gonelli A, Carnevale E, et al. TRAIL promotes the survival and proliferation of primary human vascular endothelial cells by activating the Akt and ERK pathways. Circulation 2003; 107 (17):2250-6.
39. Saeki T, Mhashilkar A, Swanson X, et al. Inhibition of human lung cancer growth following adenovirus-mediated mda-7 gene expression in vivo. Oncogene 2002; 21 (29):4558-66.

What is claimed:

1. A composition comprising a recombinant adeno-associated virus 2/5 (rAAV2/5) which encodes consecutive amino acids 114-281 of the amino acid sequence of the extracellular domain of tumor necrosis factor-related apoptosis-inducing ligand (TRAIL).

2. The composition of claim 1, wherein the consecutive amino acids 114-281 of the amino acid sequence of the extracellular domain of TRAIL is a rat TRAIL sequence.

3. The composition of claim 1, wherein the consecutive amino acids 114-281 of the amino acid sequence of the extracellular domain of TRAIL is a human TRAIL sequence.

4. A pharmaceutical composition comprising the composition of claim 1 and a pharmaceutically acceptable carrier.

5. A method of inducing apoptosis in a cell comprising introducing into the cell the composition of claim 1.

6. The method of claim 5, wherein the cell is a lung cancer tumor cell.

7. A method of inhibiting tumor cell growth comprising introducing into the cell the composition of claim 1.

8. The method of claim 7, wherein the cell is a lung cancer tumor cell.

9. A method for treating lung cancer in a subject comprising administering to the subject a therapeutically effective amount of the pharmaceutical composition of claim 4.

10. The method of claim 9, wherein the composition transduces lung tumor cells.

11. The method of claim 10, wherein the transduction results in secretion of soluble TRAIL (sTRAIL) and induces apoptosis of the lung tumor cells.

12. The method of claim 10, wherein the transduction results in high expression of soluble TRAIL (sTRAIL) in vivo.

13. The method of claim 10, wherein the transduction results in the suppression or inhibition of tumor growth.

14. The method of claim 9, wherein the pharmaceutical composition is administered invasively or non-invasively.

15. The method of claim 9, wherein the subject is human.

16. The method of claim 9, further comprising administering chemotherapeutic agents or irradiation, thereby increasing the apoptotic effect of TRAIL.

14. A kit comprising the pharmaceutical composition of claim 4, and instructions for use.
